(19) United States
(12) Patent Application Publication
(10) Pub. No.: US 2008/0119501 A1
(43) Pub. Date: May 22, 2008

(54) IMMEDIATE RELEASE OXYMORPHONE COMPOSITIONS AND METHODS OF USING SAME

(76) Inventor: William A. Hein, Chadds Ford, PA (US)

Correspondence Address: MAYER BROWN LLP, P.O. BOX 2828, CHICAGO, IL, 60690, US

(21) Appl. No.: 11/742,206

(22) Filed: Apr. 30, 2007

Related U.S. Application Data
(60) Provisional application No. 60/745,995, filed on Apr. 28, 2006.

(51) Int. Cl. A61K 31/485; A61P 25/00

(52) U.S. Cl. 514/282

(57) ABSTRACT

The present invention relates to compositions comprising oxymorphone or a pharmaceutically acceptable salt thereof and to methods of using such compositions to treat various conditions or disorders.

### RELATED CASE INFORMATION

This application claims priority to U.S. Provisional Application No. 60/745995 filed on Apr. 28, 2006, which is hereby incorporated by reference in its entirety to the extent permitted by law.

### FIELD OF THE INVENTION

The present invention relates generally to pharmaceutical compositions comprising oxymorphone or pharmaceutically acceptable salts thereof, and to methods of using such compositions to treat various conditions, diseases and disorders.

### BACKGROUND OF THE INVENTION

Oxymorphone HCL, a semisynthetic μ-opioid agonist, is known to produce more rapid onset of action and greater potency than its parent compound, morphine. Until recently, oxymorphone has only been available in suppository and intravenous formulations. If oral formulations of oxymorphone and its pharmaceutically acceptable salts could be developed, a significant advance in the art would result.

### SUMMARY OF THE INVENTION

In various embodiments, the present invention provides pharmaceutical compositions comprising oxymorphone or a pharmaceutically acceptable salt thereof. In one embodiment, the composition provides rapid onset of therapeutic effect of the oxymorphone.

In another embodiment, the present invention provides orally deliverable pharmaceutical compositions comprising an effective amount of oxymorphone or pharmaceutically acceptable salt thereof and one or more pharmaceutically acceptable excipients. The term “effective amount,” as used herein refers to an amount of oxymorphone or salt thereof that is sufficient to elicit the required or desired response, as the particular administration context may require.

These and other embodiments of the present invention are described in more detail herein below.

### DESCRIPTION OF THE DRAWINGS

FIG. 1 shows mean single-dose and steady-state plasma concentrations of oxymorphone immediate release (IR) for doses of: (a) 5 mg; (b) 10 mg; and (c) 20 mg in 23 healthy study participants.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” may mean ±5%, or ±10%, or ±20%, or ±30% depending on the relevant technology. Also, the disclosure of ranges is intended as a continuous range including every value between the minimum and maximum values recited as well as any ranges that can be formable thereby.

It is also to be understood that any ranges, ratios, and ranges of ratios that can be formed by any of the numbers or data present herein represent further embodiments of the present invention. This includes ranges that can be formed that do or do not include a finite upper and/or lower boundary. Accordingly, the skilled person will appreciate that such ratios, ranges, ranges of ratios and values are unambiguously derivable from the data and numbers presented herein.

### Oxymorphone

In one embodiment, a composition of the invention comprises oxymorphone or a pharmaceutically acceptable salt thereof, for example oxymorphone hydrochloride. “Pharmaceutically acceptable salts,” or “salts,” include salts of oxymorphone prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

In one embodiment, acid addition salts are prepared from the free base forms using conventional methodology involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like.

In other embodiments, an acid addition salt is reconverted to the free base by treatment with a suitable base. In a further embodiment, the acid addition salts are halide salts, which are prepared using hydrochloric or hydrobromic acids. In still other embodiments, the basic salts are alkali metal salts, e.g., sodium salt.

### Pharmaceutical Excipients

In one embodiment, compositions of the invention comprise one or more additional pharmaceutically acceptable excipients. The term “pharmaceutically acceptable excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition, and that does not produce unacceptable toxicity or interaction with other components in the composition.

Compositions of the invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; polyvinylpyrrolidone; and the like. Such diluents, if present, constitute in total about 5% to about 99%, about 10% to about 85%, or about 20% to about 80%, of the total weight of the composition.

Compositions of the invention optionally comprise one or more pharmaceutically acceptable disintegrants as excipients. Suitable disintegrants include, either individually or in combination, starches, including sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, xanthan, locust bean, karaya, pectin and tragacanth gums.

Disintegrants may be added at any suitable step during the preparation of the composition, particularly prior to a granulation step or during a lubrication step prior to compression. Such disintegrants, if present, typically comprise in total about 0.2% to about 30%, about 0.2% to about 10%, or about 0.2% to about 5%, of the total weight of the composition.

Compositions of the invention optionally comprise one or more antioxidants. Illustrative antioxidants include sodium ascorbate, vitamin E (tocopherol). One or more antioxidants, if present, are typically present in a composition of the invention in an amount of about 0.001% to about 5%, about 0.005% to about 2.5%, or about 0.01% to about 1%, by weight.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, about 0.75% to about 15%, or about 1% to about 10%, of the total weight of the composition.

Compositions of the invention optionally comprise one or more pharmaceutically acceptable wetting agents as excipients. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, about 0.4% to about 10%, or about 0.5% to about 5%, of the total weight of the composition.

Compositions of the invention optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium (magnesium stearate), calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, about 0.2% to about 8%, or about 0.25% to about 5%, of the total weight of the composition.

Suitable anti-adherents include talc, cornstarch, DL-leucine, sodium lauryl sulfate and metallic stearates. Talc is a anti-adherent or glidant used, for example, to reduce formulation sticking to equipment surfaces and also to reduce static in the blend. Talc, if present, constitutes about 0.1% to about 10%, about 0.25% to about 5%, or about 0.5% to about 2%, of the total weight of the composition. Glidants can be used to promote powder flow of a solid formulation. Suitable glidants include colloidal silicon dioxide, starch, talc, tribasic calcium phosphate, powdered cellulose and magnesium trisilicate.

Compositions of the present invention can comprise one or more flavoring agents, sweetening agents, and/or colorants. Flavoring agents useful in the present invention include, without limitation, acacia syrup, alitame, anise, apple, aspartame, banana, Bavarian cream, berry, black currant, butter, butter pecan, butterscotch, calcium citrate, camphor, caramel, cherry, cherry cream, chocolate, cinnamon, citrus, citrus punch, citrus cream, cocoa, coffee, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, MagnaSweet®, maltol, mannitol, maple, menthol, mint, mint cream, mixed berry, nut, orange, peanut butter, pear, peppermint, peppermint cream, Prosweet® Powder, raspberry, root beer, rum, saccharin, safrole, sorbitol, spearmint, spearmint cream, strawberry, strawberry cream, stevia, sucralose, sucrose, Swiss cream, tagatose, tangerine, thaumatin, tutti fruitti, vanilla, walnut, watermelon, wild cherry, wintergreen, xylitol, and combinations thereof, for example, anise-menthol, cherry-anise, cinnamon-orange, cherry-cinnamon, chocolate-mint, honey-lemon, lemon-lime, lemon-mint, menthol-eucalyptus, orange-cream, vanilla-mint, etc.

Sweetening agents that can be used in the present invention include, for example, acesulfame potassium (acesulfame K), alitame, aspartame, cyclamate, cylamate, dextrose, isomalt, MagnaSweet®, maltitol, mannitol, neohesperidine DC, neotame, Prosweet® Powder, saccharin, sorbitol, stevia, sucralose, sucrose, tagatose, thaumatin, xylitol, and the like.

Flavoring agents, sweetening agents, and/or colorants can be present in compositions of the invention in any suitable amount, for example about 0.01% to about 10%, about 0.1% to about 8%, or about 1% to about 5%, by weight.

The foregoing excipients can have multiple roles as is known in the art. For example, starch can serve as a filler as well as a disintegrant. The classification of excipients above is not to be construed as limiting in any manner. Excipients categorized in any way may also operate under various different categories of excipients as will be readily appreciated by one of ordinary skill in the art.

### Pharmaceutical Dosage Forms

Compositions of the present invention can be formulated as solid, liquid or semi-solid dosage forms. In one embodiment, such compositions are in the form of discrete dose units or dosage units. The terms “dose unit” and/or “dosage unit” herein refer to a portion of a pharmaceutical composition that contains an amount of a therapeutic agent suitable for a single administration to provide a therapeutic effect. Such dosage units may be administered one to a small plurality (i.e. 1 to about 4) of times per day, or as many times as needed to elicit a therapeutic response. A particular dosage form can be selected to accommodate any desired frequency of administration to achieve a specified daily dose. Typically one dose unit, or a small plurality (i.e. up to about 4) of dose units, provides a sufficient amount of the active drug (e.g. oxymorphone) to result in the desired response or effect.

In one embodiment, a single dosage unit, be it solid or liquid, comprises a therapeutically and/or prophylactically effective amount of oxymorphone or a pharmaceutically acceptable salt thereof. The term “therapeutically effective amount” or “therapeutically and/or prophylactically effective amount” as used herein refers to an amount of compound or agent that is sufficient to elicit the required or desired therapeutic and/or prophylactic response, as the particular treatment context may require.

It will be understood that a therapeutically and/or prophylactically effective amount of a drug for a subject is dependent inter alia on the body weight of the subject. A “subject” herein to which a therapeutic agent or composition thereof can be administered includes a human subject of either sex and of any age, and also includes any nonhuman animal, particularly a domestic or companion animal, illustratively a cat, dog or a horse.

### Solid Dosage Forms

In various embodiments, compositions of the invention are in the form of solid dosage forms or units. Non-limiting examples of suitable solid dosage forms include tablets (e.g. suspension tablets, bite suspension tablets, rapid dispersion tablets, chewable tablets, effervescent tablets, bilayer tablets, etc), caplets, capsules (e.g. a soft or a hard gelatin capsule), powder (e.g. a packaged powder, a dispensable powder or an effervescent powder), lozenges, sachets, cachets, troches, pellets, granules, microgranules, encapsulated microgranules, powder aerosol formulations, or any other solid dosage form reasonably adapted for oral administration.

Tablets are an illustrative dosage form for compositions of the invention. Tablets can be prepared according to any of the many relevant, well known pharmacy techniques. In one embodiment, tablets or other solid dosage forms can be prepared by processes that employ one or a combination of methods including, without limitation, (1) dry mixing, (2) direct compression, (3) milling, (4) dry or non-aqueous granulation, (5) wet granulation, or (6) fusion.

The individual steps in the wet granulation process of tablet preparation typically include milling and sieving of the ingredients, dry powder mixing, wet massing, granulation and final grinding. Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). Typically, no wet binder or moisture is involved in any of the steps.

In another embodiment, solid dosage forms such as tablets can be prepared by mixing oxymorphone with one or more optional pharmaceutical excipient to form a substantially homogeneous preformulation blend. The preformulation blend can then be subdivided and optionally further processed (e.g. compressed, encapsulated, packaged, dispersed, etc.) into any desired dosage forms.

Compressed tablets can be prepared by compacting a powder or granulation composition of the invention. The term “compressed tablet” generally refers to a plain, uncoated tablet suitable for oral ingestion, prepared by a single compression or by pre-compaction tapping followed by a final compression. Tablets of the present invention may be coated or otherwise compounded to provide a dosage form affording the advantage of improved handling or storage characteristics. Preferably, however, any such coating will be selected so as to not substantially delay onset of therapeutic effect of a composition of the invention upon administration to a subject. The term “suspension tablet” as used herein refers to a compressed tablet that rapidly disintegrates after placement in water.

In one embodiment, compositions of the invention are suitable for rapid onset of therapeutic effect, particularly with respect to the oxymorphone. In another embodiment, upon oral administration of a composition of the invention to a subject, at least a therapeutically effective amount of the oxymorphone.

In another embodiment, upon oral administration of a composition of the invention to a plurality of adult human subjects (following single dose), the subjects exhibits one or more of the following:

(a): an average Tₘₐₓ of oxymorphone within about 0.1 to about 3 hours, about 0.15 to about 2.5 hours, or about 0.2 to about 2 hours after administration;

(b) an average Cmax (μg/ml) of about 0.25 to about 6.0, about 0.5 to about 5.5, or about 1 to about 4.5; and/or

(c) an average AUC_{0-∞} (μg*h/I) of about 3 to about 25, about 4 to about 22, or about 4.25 to about 21.

Compositions of the invention are useful in the treatment of various conditions and disorders including, without limitation, pain, post-surgical pain, lower back pain, etc.

### Storage Stability

Illustrative suspension compositions of the invention comprise oxymorphone or pharmaceutically acceptable salt thereof and one or more optional pharmaceutical excipients. Such compositions, upon storage in a closed container maintained at room temperature, refrigerated (e.g. about 5 to about 5-10° C.) temperature, or frozen for a period of about 1, 2, 3, 4, 5, 6, 7, 8, 9, 10, 11, or 12 months, preferably exhibit at least about 90%, more preferably at least about 92.5%, still more preferably at least about 95%, and yet more preferably at least about 97.5% of the oxymorphone present therein.

### EXAMPLES

### Example 1

Oxymorphone HCL immediate release tablets were prepared in 5 mg and 10 mg strengths as show in Tables 1 and 2.

TABLE 1

| | | Tablet | Exhibit Batch |
|---|---|---|---|
| | | Composition | (kg/batch) |
| Ingredient | Function | (mg/tablet) | 33 kg, 150,000 tablets |
| Oxymorphone | Active | 5 | 0.75 |
| HCL | Ingredient | | |
| Lactose | Filler | 197.40 | 29.61 |
| monohydrate | | | |
| Pregelatinized | Disintegrant | 15.4 | 2.310 |
| starch | | | |
| Magnesium | Lubricant | 1.65 | 0.2475 |
| stearate | | | |
| FD&C Blue #2 | Color | 0.55 | 0.0825 |
| Total | | 220 | 33.0 |

TABLE 2

| | | Tablet | Exhibit Batch |
|---|---|---|---|
| | | Composition | (kg/batch) |
| Ingredient | Function | (mg/tablet) | 33 kg, 150,000 tablets |
| Oxymorphone | API | 10 | 1.5 |
| HCL | | | |
| Lactose | Filler | 191.45 | 28.72 |
| monohydrate | | | |
| Pregelatinized | Disintegrant | 15.4 | 2.310 |
| starch | | | |
| Magnesium | Lubricant | 1.65 | 0.2475 |
| stearate | | | |
| FD&C Blue #2 | Color | 1.50 | 0.2250 |
| Total | | 220 | 33.0 |

### Example 1

### Efficacy of Oxymorphone Immediate Release for Moderate to Severe Pain After Abdominal Surgery: A Randomized, Placebo-Controlled, Multiple-Dose Trial

Introduction: The current multidose trial of oxymorphone immediate release (IR) assessed pain relief in patients with moderate to severe pain after abdominal surgery. Oxymorphone immediate release (IR) is an oral opioid that provides rapid (tₘₐₓ=30 min), dose-proportional analgesia for moderate to severe pain after total hip or knee replacement.

Materials and Methods: Oxymorphone IR tablets were formulated as above. Patients with pain 50 mm on 100-mm visual analog scale within 30 hours of abdominal surgery were randomized to receive double-blind oxymorphone IR 10 mg, oxymorphone IR 20 mg, oxycodone IR 15 mg, or placebo q4-6 h. For first-dose evaluations, the primary end point was sum of pain intensity difference (SPID); pain intensity and relief were measured periodically within 6 hours of the first dose. For the 48-hour multiple-dose period, time to study discontinuation was the primary end point, and pain intensity was measured before each dose. This study had IRB approval, and patients provided written informed consent.

Results: The randomized population included 331 patients (99% women; mean±SD age, 42.6±9.3 y). In the single-dose period, the least square mean SPID categorical score was statistically significantly higher in oxymorphone IR 10-mg (3.1), oxymorphone IR 20-mg (3.9), and oxycodone IR (3.3) groups compared with placebo (1.7; P<0.05). In the multiple-dose phase, the median [95% CI] time to discontinuation (h) was significantly longer for oxymorphone IR 10 mg (17.9 [4.532.6]), oxymorphone IR 20 mg (20.3 [6.0-48.0]), and oxycodone IR (24.1 [5.0-48.0]) than for placebo (4.8 [3.4-7.5]; P<0.01). Patient ratings were superior for oxymorphone IR vs placebo (P<0.02). Adverse events in >5% of opioid-treated patients were nausea, vomiting, headache, dizziness, and pruritus.

Conclusions: Oxymorphone IR, given every 4-6 hours over 2 days, provided adequate pain relief for moderate to severe pain following lower abdominal surgery, with a safety and tolerability profile expected for oral opioid analgesics.

### Example 2

### Oxymorphone Immediate Release for Postsurgical Abdominal Pain: A Single-and Multiple-Dose Randomized, Placebo-Controlled, Active-Comparator Trial

Oxymorphone immediate release (IR) (formulated as above) is a μ-opioid receptor agonist formulation that achieves maximum plasma concentration within 30 minutes' while providing efficacious analgesia following orthopedic surgery. In this study, patients were randomized to receive oxymorphone IR (10 or 20 mg), oxycodone IR 15 mg, or placebo (q4-6 h up to 48 h) for moderate to severe pain (pain intensity of 50 mm on a 0-100-mm Visual Analog Scale) during the 30 hours following abdominal surgery. The primary efficacy parameter was time to discontinuation during 48 hours of treatment. End points in the single-dose phase (0-6 h) included sum of pain intensity differences (SPID) and pain relief (categorical scales). Of the 331 treated patients, 99% were women, and the mean (standard deviation) age was 42.6 (9.3) years. The median (95% confidence interval) time (h) to discontinuation in the multiple-dose phase was 17.9 (4.5-32.6), 20.3 (6.0-48.0), 24.1 (5.0-48.0), and 4.8 (3.4-7.5) for oxymorphone IR 10 mg, oxymorphone IR 20 mg, oxycodone IR, and placebo groups, respectively (P<0.05 for all opioid groups vs placebo). In the single-dose phase, the least squares (LS) mean SPID score was significantly higher in the oxymorphone IR 10-mg (3.1), oxymorphone IR 20-mg (3.9), and oxycodone IR (3.3) groups versus placebo (1.7; P<0.05). The LS mean total pain relief was significantly higher in the oxymorphone 20-mg group (11.7) compared with placebo (8.2; P<0.001). Adverse events occurring at >5% were comparable between active treatment groups; generally mild to moderate; and included nausea, vomiting, headache, dizziness, and pruritus. In conclusion, these results show that oxymorphone IR given every 4 to 6 hours can provide rapid, effective, and tolerable analgesia for moderate to severe postsurgical pain as either a single- or multiple-dose regimen.

### Example 3

### Single- and Multiple-Dose Pharmacokinetic and Dose-Proportionality Study of Oxymorphone Immediate-Release Tablets

The objective of the current study was to elucidate the pharmacokinetics and dose proportionality of oxymorphone and its two metabolites (6-OH-oxymorphone and oxymorphone-3-glucuronide) following single- and multiple-dose administration of a new formulation of oxymorphone hydrochloride IR tablets formulated as above.

### Study Design and Methods.

Study Design. This study used a randomised three-way crossover design. Assuming a maximum multiplicative coefficient of variation ≦30% for the AUC, a sample size of 18 subjects was estimated to provide 80% power to detect a 20% difference in pharmacokinetic variables between two dose levels. The subject sample size was increased to 24 to account for dropouts and enhance the strength of the results.

Methods. This study was conducted in compliance with the International Conference on Harmonisation and applicable FDA regulations. Before initiating a 14-day screening period, the investigators informed each prospective subject of the nature of the study, explained the potential risks, and obtained written informed consent. Screening evaluations for all subjects consisted of a medical history and review of systems, medication history, physical examination (including routine vital signs), 12-lead electrocardiogram, laboratory evaluations, bodyweight, urine drug screen, and hepatitis and human immunodeficiency virus (HIV) testing.

Eligibility Criteria. Healthy men and nonpregnant/nonlactating women between 18 and 45 years of age who were nonsmokers and of normal bodyweight (i.e. ≧50 to ≦100 kg and within 15% of standard weight using the Metropolitan Life Insurance Company's standards) were recruited. Women were required to use a medically acceptable form of birth control for the study duration and to have a negative pregnancy test before each treatment period. Alcoholic beverages were not permitted beginning 72 hours before administration of the first dose of study medication. Additional exclusion criteria included prior oxymorphone IR (Endo Pharmaceuticals Inc., Chadds Ford, Pa., USA) exposure; known allergy to oxymorphone or naltrexone hydrochloride (ReVia®, DuPont Pharmaceuticals, Wilmington, Del., USA), a positive screen for hepatitis B, hepatitis C or HIV; a history of alcohol abuse, physical dependence on any opioid, or drug abuse or addiction; or a positive urine drug screen for ethanol, cocaine, tetrahydrocannabinol, barbiturates, amphetamines, benzodiazepines or opiates. At screening and on admission, subjects with clinically significant laboratory abnormalities or significant disease as determined by physical examination and laboratory analysis were excluded.

Study Drug Administration. Eligible subjects were to receive all three dose levels of oxymorphone IR tablets (5, 10 and 20 mg [two 10 mg tablets]) during three separate treatment periods, with the timing based on a nonblinded randomisation schedule that was balanced for sequence and period. On day 1 of each treatment period, subjects received a single dose of oxymorphone IR 5, 10 or 20 mg in the morning. On day 3 through to the morning of day 8, subjects received this same dose of oxymorphone every 6 hours, for a total of 22 doses per 8-day treatment period. Subjects received a single 50 mg dose of the opioid antagonist naltrexone hydrochloride each evening on days −1 to 7 as protection against opioid-related adverse events. Each subject received 240 mL of drinking water with every dose. After each treatment period, a 7-day washout was instituted.

On days 1 and 8, subjects fasted from approximately 10:00 pm in the evening until 4 hours after administration of the morning oxymorphone IR dose (given at approximately 8:00 am), after which time a standard luncheon was provided. On days 2 through 7, a moderate- to low-fat breakfast (e.g. fat calories 530% of total calories) was allowed. On days 3 through 7, breakfast was served 1 hour after administration of the morning oxymorphone IR dose. Xanthine-containing foods and beverages were prohibited throughout the treatment periods.

Subjects were instructed to avoid taking concommitant medications within 2 weeks before enrolment for prescription medication and within 4 weeks for prescription medication known to potentially affect hepatic metabolism. Nonprescription medications were not allowed within 24 hours before enrolment. Investigators were permitted to authorise the short-term use of a nonprescription medication for a self-limiting indication (e.g. aspirin for headache).

Each subject's eligibility status was confirmed (including urine drug and pregnancy testing) on the day before the planned start of each treatment period. Any subject who received study medication was to remain in the trial and complete all required tests and evaluations, provided they did not enter the study in violation of the protocol, (or be withdrawn based on investigator discretion), or develop an exclusion criterion and/or a requirement for an unacceptable concomitant medication.

Plasma Sampling Schedule. During each of the three treatment periods, venous blood samples were collected to determine the single-dose profile (collection at 0 [predose], 0.25, 0.5, 1, 1.5, 2, 3, 4, 6, 8, 10, 12, 24, 36 and 48 hours after single-dose administration on the morning of day 1) and the steady-state profile (collection at 0 hours and at 0.25, 0.5, 1, 1.5, 2, 3, 4 and 6 hours after the morning dose on day 8).

Safety Assessments. Participants remained in the clinic during each 8-day treatment period and were carefully monitored for the development of adverse events. Physical examination and a series of laboratory tests were performed at the conclusion of the final treatment period, and routine vital signs (including pulse, respiratory rate and blood pressure) were obtained each morning throughout the three treatment periods.

Analytical Methods. Plasma concentrations of oxymorphone, 6-OHoxymorphone, and oxymorphone-3-glucuronide were determined with parallel high-performance liquid chromatography (HPLC) combined with tandem mass spectrometry (parallel LC/MS/MS). Two methods have been developed and validated by SFBC Analytical Laboratories, Inc. (North Wales, Pa., USA). Oxymorphone and 6-OH-oxymorphone concentrations were determined following a liquid-liquid extraction from alkalinised human plasma: 50 μL of 50% acetonitrile (v/v), 50 μL of d3-oxymorphone and d3-6-OH-oxymorphone internal standards, and 1 mL of 0.2N ammonium hydroxide were added to 0.5 mL of plasma. After briefly vortexing, samples received 4 mL of methyl-t-butyl ether and were again vortexed (2 minutes) and the phasesseparated by centrifugation. Three millilitres of the organic phase were transferred to a new tube, evaporated under nitrogen in a 40° C. water bath, and reconstituted in 150 μL of HPLC mobile phase (50% acetonitrile/50% of 0.1% formic acid in water). A 20 μL aliquot was injected into a SCIEX API 3000 series LC/MS/MS system (MDI SCIEX, Concord, ON, Canada). The linear range of quantitation for oxymorphone and 6-OH-oxymorphone was 0.1-20 (μg/L plasma.

Oxymorphone-3-glucuronide concentrations were determined following solid-phase extraction from acidified human plasma: 100 μL of d3-oxymorphone-3-glucuronide internal standard and 2 mL of 0.1% trifluoroacetic acid (TFA) were added to 0.5 mL of plasma. Samples were vortexed and centrifuged, and the supernatant was applied to a solid-phase extraction column. Columns were first equilibrated by successive 3 mL washes with methanol, water and 0.1% TFA. After sample application, columns were washed with 2 mL of 0.1% TFA, dried under a vacuum for 60 seconds, and eluted with 3 mL of 50% methanol (v/v). The eluant was dried under nitrogen in a 55° C. water bath and reconstituted in 150 μL of HPLC mobile phase (90% acetonitrile/0.88% formic acid in water), and 20 μL was injected into the LC/MS/MS system (using a PE SCIEX API 300). The linear range of quantification for oxymorphone-3-glucuronide was 5-250 μ/L of plasma. Least squares linear regression modeling was performed to calculate the calibration curves.

Specificity of the procedures was established by determining the chromatographic profiles of purified standards of oxymorphone, 6-OH-oxymorphone, and oxymorphone-3-glucuronide as well as for d3-labeled analytes, and then ensuring that (i) there was no interference from control plasma, and (ii) the chromatographic profile of each analyte (including naltrexone) did not interfere with the other analytes. For example, the oxymorphone-3-glucuronide standard and the d3-labeled compound produced peaks with mass to charge ratios (m/z) at 478.4→284 m/z and 481.4—s 287 m/z, respectively. These peaks were not produced by control plasma or by the other analytes, including naltrexone.

TABLE 3
Calculation of pharmacokinetic variables

| | |
|---|---|
| C/LF | Oral clearance; calculated as dose/AUO- or dose/AUC_{SS} |
| Cₘₐₓ | Maximum plasma concentration; the highest concentration |
| | observed during a dosage interval |
| Cₘᵢₙ | Minimum plasma concentration; the concentration measured just |
| | before dose administration |
| tₘₐₓ | The time that Orr., was observed |
| λ_{z} | The terminal elimination rate constant; calculated using linear |
| | regression on the terminal portion of the La-concentration vs |
| | time curve |
| t½ | Terminal elimination half-life: calculated as 0.693/λ_{z} |
| AUC_{∞} | Area under the plasma concentration vs time curve from time 0 |
| | to infinity: calculated as AUC₁ + C/λ_{z} |
| AUC_{SS} | Area under the plasma concentration versus time curve from |
| | time 0 to the end of 1 dosage interval at steady state (i.e. |
| | time 0-c); calculated using linear trapezoid rule |
| C_{avg} | Average plasma concentration; calculated as AUO_{SS}/r |

Intraday precision (percentage coefficient of variation) was 1.43-3.93, 2.12-7.87 and 1.39-6.79 for oxymorphone, 6-hydroxy-morphone and oxymorphone-3-glucuronide, respectively. The percentage accuracy for oxymorphone, 6-hydroxymorphone and oxymorphone-3-glucuronide was 94.33-96.56, 98,17-102.56 and 101.00-105.31, respectively.

Statistical Methods. Plasma pharmacokinetic variables were calculated from the concentration data using standard noncompartmental methods (Table I). Relationships between the pharmacokinetic variables and dose were explored and summarised using appropriate descriptive statistics, with assessment of the significance of main effects and interactions at the 0.10 level. Continuous variables were compared using analysis of variance (ANOVA) with dose, period, sequence and subject (sequence) included in the model using PROC GLM of SAS version 8.2 (Cary, N.C., USA). Dose proportionality was assessed after normalising the AUC from time zero to infinity (AUC_{∞}) and maximum plasma concentration (Cₘₐₓ) values to a 10 mg dose. Steady-state conditions were confirmed by analysis of mean trough(predose) concentrations, and the 95% confidence intervals (CIs), on days 6 through 8.

Adverse events were reported in terms of severity (mild, moderate or severe) and relationship to study medication (unlikely, possibly or probably), and the incidence of adverse events was compared across treatment groups using an appropriate nonparametric statistic. All laboratory results, vital signs measurements, and other safety variables were summarised using mean values and changes from baseline.

### Results.

Study Population. Twenty-four healthy subjects were enrolled; 23 completed the study. One subject withdrew consent to participate in the trial and was not evaluated for phartnacokinetics. Table II provides the characteristics for all enrolled subjects. The study population was unremarkable except that the majority of subjects had a large body frame.

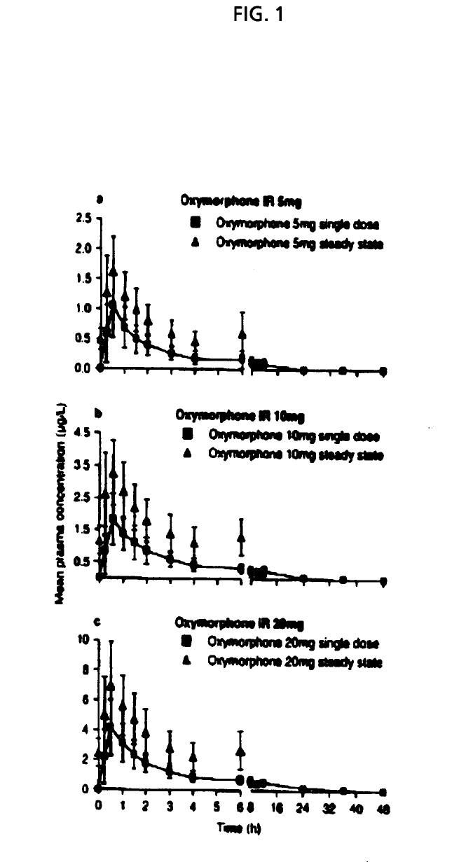

Single-Dose Pharmacokinetics. After single-dose administration of oxymorphone IR, mean plasma concentrations and calculated pharmacokinetic parameters (AUC_{∞} and Cₘₐₓ) for oxymorphone increased proportionally as the dose was increased from 5 to 10 mg and from 10 to 20 mg (table III). The plasma concentrations of the metabolites also increased with each doubling of dose. The elimination half-life (t_{1/2},) for oxymorphone administered through this new formulation was approximately 7.3-9.4 hours. The mean plasma concentration versus time curves for single-dose oxymorphone are depicted in FIG. 1. Similar curves for the metabolites were also observed (data not shown).

TABLE 4
Study participant characteristics (n-24)

| | | |
|---|---|---|
| Mean age [y] (range) | 36 | (27-44) |

Sex [n (%)]^{a}

| | | |
|---|---|---|
| male | 12 | (50) |
| female | 12 | (50) |

Ethnic group [n (°/0)]

| | | |
|---|---|---|
| White | 16 | (67) |
| Hispanic | 7 | (29) |
| Black | 1 | (4) |
| Mean weight [kg] (range) | 71.3 | (54.5-100) |
| Mean height [cm] (range) | 164.8 | (149.9-185) |

Frame In (%)]

| | | |
|---|---|---|
| small | 1 | (4) |
| medium | 6 | (25) |
| large | 17 | (71) |

^{a}Twelve men (52%) and 11 women (48%) completed the study.
^{b}Frame size was determined by elbow breadth as detailed by the Metropolitan Life Insurance Company tables.

TABLE 5
Pharmacokinetic properties of single doses of oxymorphone IR in 23 healthy
individuals^{e}

| | Oxymorphone | | | | | |
|---|---|---|---|---|---|---|
| | IR dose | | | | | |
| | 5 mg | | 10 mg | | 20 mg | |
| Oxymorphone | | | | | | |
| AUC.. (μg · h/L) | 4.48 | (2.07) | 9.10 | (3.40) | 20.07 | (5.80) |
| Cmax (μg/L) | 1.10 | (0.55) | 1.93 | (0.75) | 4.39 | (1.72) |
| tₘₐₓ (h) | 0.50 | (0.25-1 AO)″ | 0.50 | (0.25-1.50)° | 0.50 | (0.25-1.00)° |
| CL/F (L/min) | 23.53 | (13.18) | 21.42 | (9.92) | 18.21 | (6.16) |
| [symbol] (h⁻¹) | 0.1534 | (0.1308) | 0.1257 | (0.0997) | 0.0835 | (0.0331) |
| t_{1/2} (h) | 7.25 | (4.40) | 7.78 | (3.58) | 9.43 | (3.36) |
| 6-OH-oxymorphone | | | | | | |
| AUC_{∞}. (μg · h/L) | 4.02 | (3.18) | 9.90 | (5.13) | 24.37 | (10.50) |
| Cₘₐₓ (μg/L) | 095 | (0.52) | 1.62 | (0.75) | 3.57 | (1.41) |
| tₘₐₓ (h) | 0.50 | (0.25-1.00)^{b} | 0.50 | (0.50-1.50)′ | 0.50 | (0.25-1.00)^{b} |
| [symbol] (h−¹) | 0.1661 | (0.1677) | 0.0752 | (0.0678) | 0.0414 | (0.0134) |
| t_{1/2}, (h) | 7.27 | (4.76) | 13.72 | (6.55) | 18.35 | (5.77) |
| Oxymorphone-3-g Iucuronide | | | | | | |
| AUC (μg · /hL) | 650.03 | (140.05) | 1322.72 | (261.76) | 2672.40 | (480.33) |
| Cmax (μg/L) | 134.24 | (30.02) | 265.78 | (63.24) | 516.26 | (106.53) |
| tₘₐₓ (h) | 1.00 | (1.00-1.50)^{b} | 1.00 | (1.00 2.00)^{b} | 1.00 | (1.00-1.50)° |
| [symbol] (h−¹) | 0.0936 | (0.0360) | 0.0805 | (0.0218) | 0.0763 | (0.0178) |
| t_{1/2} (h) | 8.48 | (3.11) | 9.15 | (2.18) | 9.67 | (2.71) |

^{a}Mean (SD), unless otherwise specified.
^{b}Median (range). [symbol]
= the terminal elimination rate constant;
AUC = area under the plasma concentration vs time curve from time zero to infinity;
Cₘₐₓ = maximum plasma concentration, the highest concentration observed during a dosage interval;
CL/F = oral clearance;
IR = immediate release;
Imax = the time that _{Cmax} was observed;
b/, = terminal elimination half-life.

Steady-State Pharmacokinetics. Trough concentrations derived from pre-morning-dose plasma levels after 3, 4 and 5 days of multiple-dose treatment demonstrated that steady-state conditions for oxymorphone were achieved after 3-4 days of dosing every 6 hours (table IV). Similar results were observed for the metaboliteplasma levels. As seen following single-dose administration, steady-state mean plasma concentrations (AUCₛₛ) and Cₘₐₓ of oxymorphone (table V) also rose in a linear fashion following increasing dose. In addition, the plasma concentrations of the metabolites 6-OH-oxymorphone and oxymorphone-3-glucuronide also increased approximately 2-fold with each doubling of dose. FIG. 1 illustrates the effect of 5, 10 and 20 mg doses on steady-state plasma concentrations of oxy-morphone. A similar pattern was also observed for the metabolites 6-OH-oxymorphone and oxy-morphone-3-glucuronide (data not shown).

Dose Proportionality. There were no statistically significant across-dose differences in dose-normalised AUC_{∞}, AUCₛₛ or Cₘₐₓ for oxymorphone (table VI), confirming linear and dose-proportional pharmacokinetics after single-dose administration and at steady state for the parent compound.

Safety. There were only two adverse events related to treatment reported at the 20 mg dose. One subject reported mild dry mouth; the other reported mild vomiting, nausea and dizziness that caused discontinuation on day 3. No clinically significant changes in laboratory values, vital signs, physical examination findings, or electrocardiogram readings were recorded.

Discussion. In this randomised crossover trial of oxymorphone IR in healthy volunteers, the single-dose and steady-state pharmacokinetic profiles of oxymorphone were linear and dose proportional across a dose range of 5-20 mg. Oxymorphone IR was not associated with any clinically significant effects on laboratory or other safety variables.

After single-dose administration and at steady state, the increases in mean AUC_{∞} and AUCₛₛ, respectively, and Cₘₐₓ for oxymorphone followed a 2-fold progression between doses. Mean oxymorphone AUCₛₛ, was 4.63, 10.19 and 21.10 following administration of oxymorphone IR 5, 10 and 20 mg every 6 hours, respectively. These data provide important information for the clinician in that an increase in dose in the clinical setting will produce predictable increases in the plasma levels of oxymorphone. This is particularly important for opioid drugs. Patient pain is highly individual, responses to opioids are variable, and clinicians frequently must titrate opioid drugs across a large milligram dose range to find a dosage that provides adequate analgesia with acceptable opioid-related adverse events. Formulations that provide predictable increases in drug concentrations with increasing dose provide less uncertainty when empirically determining a patient's optimum dose. Moreover, these data are similar to results recently published on the linearity and dose proportionality of oxymorphone ER.

TABLE 6
Analysis of trough plasma concentrations
for oxymorphone at steady state

| Oxymorphone | Mean (95% CI) Cmin | | |
|---|---|---|---|
| dose (mg) | day 6 | day 7 | day 8 |
| 5 | 0.47 (0.40, 0.53) | 0.50 (0.44, 0.57) | 0.49 (0.42, 0.55) |
| 10 | 0.98 (0.82, 1.14) | 0.99 (0.83, 1.15) | 1.16 (1.00, 1.32) |
| 20 | 2.05 (1.71, 2.40) | 2.31 (1.96, 2.65) | 2.47 (2.21, 2.81) |

Cmin = minimum plasma concentration in μg/L

TABLE 7
Steady-state pharmacokinetic properties of oxymorphone
IR administered 6-hourly in 23 healthy individuals'

| Oxymorphone IR dose | | |
|---|---|---|
| 5 mg q6 h | 10 mg q6 h | 20 mg q6 h |

Oxymorphone

| | | | | | | |
|---|---|---|---|---|---|---|
| AUC_{SS} (μg · h/L) | 4.63 | (1.49) | 10.19 | (3.34) | 21.10 | (7.59) |
| Cₘₐₓ (μg/L) | 1.73 | (0.62) | 3.51 | (0.91) | 7.33 | (2.93) |
| Cₘᵢₙ (μg/L) | 0.49 | (0.17) | 1.16 | (0.43) | 2.47 | (0.94) |
| tₘₐₓ (h) | 0.50 | (0.25-6.00)^{b} | 0.50 | (0.25-1.00)^{b} | 0.50 | (0.25-1.50)' |
| C_{avg} (μg/L) | 0.77 | (0.25) | 1.70 | (0.56) | 3.52 | (1.27) |
| C/LF (L/min) | 19.23 | (4.27) | 17.63 | (4.42) | 17.68 | (6.14) |

6-OH-oxymorphone

| | | | | | | |
|---|---|---|---|---|---|---|
| AUC_{SS} (μg · h/L) | 4.98 | (2.07) | 10.77 | (4.21) | 23.68 | (10.14) |
| Cₘₐₓ (μg/L) | 1.55 | (0.52) | 3.12 | (1.08) | 6.94 | (2.86) |
| Cₘᵢₙ (μg/L) | 0.70 | (0.33) | 1.52 | (0.71) | 3.34 | (1.45) |
| tₘₐₓ (h) | 0.50 | (0.25-0.50)^{b} | 0.50 | (0.25-1.00)^{b} | 0.50 | (0.25-1.50)^{b} |
| C_{avg} (μg/L) | 0.83 | (0.35) | 1.80 | (0.70) | 3.95 | (1.69) |

Oxymorphone-3-glucuronide

| | | | | | | |
|---|---|---|---|---|---|---|
| AUC_{SS} (μg · h/L) | 618.77 | (114.59) | 1321.37 | (226.44) | 2557.29 | (463.87) |
| Cₘₐₓ | 180.25 | (32.96) | 376.25 | (69.62) | 722.24 | (143.62) |
| Cₘᵢₙ (μg/L) | 59.67 | (14.75) | 136.77 | (31.32) | 272.54 | (61.04) |
| tₘₐₓ (h) | 1.00 | (0.50-1.50)^{b} | 1.00 | (1.00-1.50)^{b} | 1.00 | (1.00-2.00)^{b} |
| C_{avg} (μg/L) | 103.13 | (19.10) | 220.23 | (37.74) | 426.22 | (77.31) |

^{a}Mean (SD), unless otherwise specified.
^{b}Median (range).
AUC_{SS} = area under the plasma concentration versus time curve from time 0 to the end of 1 dosage interval at steady state; C_{avg} = average plasma concentration; CUF = oral clearance; Cₘₐₓ = maximum plasma concentration, the highest concentration observed during a dosage interval; Cm,. = minimum plasma concentration; IR = immediate release; q6 h = every 6 hours; tₘₐₓ = the time that Cₘₐₓ was observed.

TABLE 8
Dose proportionality following a single dose
or at steady state, normalised to 10 mg^{e}

| Oxymorphone IR dose | | | |
|---|---|---|---|
| 5 mg | 10 mg | 20 mg | p-Value |

Single dose
Oxymorphone

| | | | | |
|---|---|---|---|---|
| Ln-AUC | 8.09 (1.05) | 8.49 (1.05) | 9.65 (1.05) | 0.0627 |
| (μg · IA) | | | | |
| Ln-Cₘₐₓ | 2.00 (1.06) | 1.80 (1.06) | 2.04 (1.06) | 0.2124 |
| (μg/L) | | | | |
| Ln-CL/F | 20.61 (1.05) | 19.62 (1.05) | 17.27 (1.05) | 0.0627 |
| (L/min) | | | | |

Steady state
Oxymorphone

| | | | | |
|---|---|---|---|---|
| Ln-AUC_{SS} | 8.91 (1 03) | 9.76 (1.03) | 9.97 (1.03) | 0.0600 |
| (μg · h/L) | | | | |
| Ln-Cₘₐₓ | 3.31 (1.05) | 3.41 (1.05) | 3.44 (1.05) | 0.8338 |
| (μg/L) | | | | |
| Ln-CL/F | 18.70 (1.03) | 17.08 (1.03) | 16.72 (1.03) | 0.0600 |
| (L/min) | | | | |

^{a}Geometric least square means (standard error).
^{b}Every 6 hours (a total of 22 doses).
AUC = area under the plasma concentration vs time curve; AUC_{SS} = area under the plasma concentration vs time curve from time 0 to the end of 1 dosage interval at steady state; CUF = oral clearance; Cm. = maximum plasma concentration, the highest concentration observed during a dosage interval; IR = immediate release; Ln = natural log-transformed data for each indicated variable.

A limitation of this study is that the plasma concentrations for 6-OH-oxymorphone at the 5 and 10 mg dose were below the lower limit of quantification in some samples and may account for some deviation in linearity that was observed with the 6-OH metabolite. In support of this interpretation, we note that plasma concentrations for 6-OH-oxymorphone were higher and, therefore, more reliably ascertained at steady state than during single-dose administration. Accordingly, the steady-state concentrations of 6-OH-oxymorphone exhibited less deviation from a linear dose response (approximately 10%) than during single-dose administration (approximately 20%).

An important pharmacokinetic parameter for an IR analgesic is a short time to peak plasma concentration. In the present study, the median time to C max (tₘₐₓ) was 0.5 hours across all doses of oxymorphone IR. This value compares favourably with IR tablets of morphine and oxycodone, for which mean tₘₐₓ has been reported to be 1.2 and 1.5 hours, respectively. Although the tₘₐₓ of oxymorphone predicts a more rapid onset of analgesia, the t½, of oxymorphone IR was measured at 7.25-9.43 hours, considerably longer than the 3- to 5.7-hour values reported for oxycodone. However, active-controlled clinical trials will be required to determine whether the shorter tₘₐₓ and longer V!, of oxymorphone IR translate into more rapid and sustained analgesia compared with other short-acting opioids.

Conclusions. After single- and multiple-dose administration of oxymorphone IR at doses ranging from 5 to 20 mg, there was a linear and dose-proportional increase in pharmacokinetics for oxymorphone. From the perspective of the clinician, this study underscores the notion that increasing doses of oxymorphone IR will result in predictable increases in the plasma concentrations of oxymorphone and its metabolites.

What is claimed is:

1. An orally deliverable pharmaceutical composition comprising oxymorphone or a pharmaceutically acceptable salt thereof and a disintegrant, where upon administration of the composition to a group of subjects, the subjects exhibit one or more of the following:
(a) an average Tₘₐₓ of oxymorphone within about 0.1 to about 3 hours after administration; (b) an average Cₘₐₓ of about 0.25 to about 6.0 μg/ml; or (c) an average AUC_{0-∞} (μg*h/I) of about 3 to about 25.

2. A method of treating pain in a subject in need thereof, the method comprising administering a composition of claim 1 to the subject.

3. The composition of claim 1 wherein the observed Tₘₐₓ of oxymorphone is between about 0.1 and 1 hour.

4. The composition of claim 1 wherein the observed average t_{1/2} of oxymorphone is greater than about 7 hours.

5. The composition of claim 3 wherein the observed average t_{1/2} of oxymorphone is greater than about 7 hours.
